(19) United States
(12) Patent Application Publication
(10) Pub. No.: US 2026/0265728 A1
(43) Pub. Date: Sep. 10, 2026

(54) LARGE-SCALE INDUCTION OF PROTEIN-PROTEIN INTERACTIONS TO DISCOVER NOVEL EFFECTOR PROTEINS FOR PROXIMITY-BASED THERAPEUTICS

(71) Applicant: THE CHILDREN'S HOSPITAL OF PHILADELPHIA, Philadelphia, PA (US)

(72) Inventors: George BURSLEM, Philadelphia, PA (US); Stephanie SANSBURY, Philadelphia, PA (US); Yevgeniy SEREBRENIK, Philadelphia, PA (US); Ophir SHALEM, Philadelphia, PA (US)

(73) Assignee: THE CHILDREN'S HOSPITAL OF PHILADELPHIA, Philadelphia, PA (US)

(21) Appl. No.: 19/166,626

(22) PCT Filed: Mar. 22, 2024
(86) PCT No.: PCT/US2024/021181

§ 371 Date: Sep. 18, 2025

Related U.S. Application Data
(60) Provisional application No. 63/492,066, filed on Mar. 24, 2023.

(51) Int. Cl. C12N 15/10

(52) U.S. Cl. CPC C12N 15/1065; C12N 15/1055

(57) ABSTRACT

Provided herein are high throughput methods of identifying a target/effector protein pair, wherein the effector protein has a desired effect on the target protein following chemical induction of dimerization. Protein-protein interactions are induced between a target of interest and any other protein using a generic heterobifunctional ligand that binds to domains introduced by pooled protein tagging. These methods provide for the high-throughput discovery of endogenous effectors for any phenotype of interest.

### REFERENCE TO RELATED APPLICATIONS

The present application claims the priority benefit of U.S. provisional application number 63/492,066, filed Mar. 24, 2023, the entire contents of which are incorporated herein by reference.

### STATEMENT OF FEDERALLY SPONSORED RESEARCH

This invention was made with government support under grant numbers CA239499, GM137416, and AG075256 awarded by the National Institutes of Health. The government has certain rights in the invention.

### BACKGROUND

### 1. Field

The present invention relates generally to the fields of molecular biology and drug discovery. More particularly, it concerns compositions and methods for tagging target endogenous proteins with ligand binding domains to allow pharmacological induction of protein-protein interactions to enable the discovery of functional effectors and effector recruitment strategies.

### 2. Description of Related Art

Over the last 20 years, the field of induced proximity therapeutics has emerged as a novel strategy to edit posttranslational modifications and expand the druggable genome beyond enzymatic targets. The most prominent and advanced type of the induced proximity strategies are applied to induce targeted protein degradation as a mechanism to completely remove disease relevant proteins from cells, tissues and organisms rather than simply inhibit their function as is the case with more traditional inhibitor-based strategies. There are two distinct, yet related, strategies for targeted protein degradation: proteolysis targeting chimera (PROTACs) and molecular glues. Both approaches rely on the induction of a lysine post-translational modification called ubiquitination. The transfer of ubiquitin results from a cascade of enzymatic reactions culminating in the formation of the lysine ubiquitin iso-peptide bond by an E3 ligase.

Both PROTACs and molecular glues rely on the recruitment of a ubiquitin E3 ligase to a non-native or neosubstrate, resulting in ectopic ubiquitination and subsequent degradation via the proteasome. The distinction between PROTACs and molecular glues lies in the fashion in which the ligase is recruited to the neo-substrate—PROTACs are heterobifunctional compounds consisting of an E3 ligase-recruiting element and a substrate-targeting element held together via a highly optimized chemical linker, whilst molecular glues are monovalent and bind to one protein, usually the E3 ligase, and alter the available protein surface such that the neo-substrate now binds despite having no measurable affinity to the molecular glue itself. Regardless of the mechanism by which the E3/neo-substrate complex is induced, the choice of the actual E3 ligase (or any other effector protein) can make the difference between success and failure₅. Target specificity, cell type specific expression, differential growth essentiality resulting in downregulation of the E3 ligase as a therapy resistance mechanism in cancer are only part of the parameters that need to be explored for the development of successful therapeutics. Unfortunately, only a handful of E3 ligases are currently in use₆ and methods for exploring additional ones are extremely limited in scale and require exogenous expression of the effector protein. Methods for using the actual endogenous effector proteins for these studies is needed to better mimic endogenous expression levels and regulation networks which will be in use when small molecule based therapeutic recruitment is developed.

### SUMMARY

As such, provided herein are methods that combine generic chemical induction of dimerization and endogenous tagging of effector proteins to systematically map effector-target relationships at large scale. These methods provide an experimental pipeline that enables testing the molecular and cellular outcome of inducing protein-protein interactions between a single protein of interest to hundreds of effector proteins.

The methods start from a cell line expressing a target protein with a known chemical binder, or a target fused to FKBP^{F36V}, for which a chemical binder already exists. Then, one of two scalable tagging technologies are used to endogenously tag hundreds of potential effector proteins in a pooled manner. In the resulting cell pool, each cell expresses the ligandable target, a single effector protein tagged with a HaloTag and an integrated molecular barcode (either an sgRNA itself or a barcode), which is used to identify the tagged effector protein. Tagging success may be investigated using flow cytometry or high-throughput imaging and in-situ barcode sequencing and identification of potential effector proteins is assessed by selecting cells with the phenotype of interest (such as, for example, reduced target protein expression, growth rate or target protein localization) and barcode identification.

In some aspects, the methods identify a target/effector protein pair, wherein the effector protein has a desired effect on the target protein following chemical induction of dimerization. The methods may comprise (i) obtaining a population of cells in which the target protein comprises or is tagged with a ligand binding domain; (ii) endogenously tagging potential effector proteins with orthogonal ligand binding domains, such that each cell in the population of cells comprises a single effector protein tagged with an orthogonal ligand binding domain and an integrated molecular barcode; (iii) contacting the population of cells with a heterobifunctional molecule that binds to both the ligand binding domain and the orthogonal ligand binding domain, thereby inducing protein-protein interactions between the target protein and the potential effector proteins; and (iv) identifying the potential effector proteins that induce the desired effect on the target protein by selecting cells with a phenotype of interest.

The ligand binding domain with which the target protein is endogenously tagged may be FKBP^{F36V}. The orthogonal ligand binding domain with which the potential effector proteins are tagged may be HaloTags, split HaloTags, or SNAP-Tags. The phenotype of interest may be the target protein expression level, altered growth rate, cell abundance, target protein disaggregation, target protein stabilization, or target protein localization.

In the methods, step (iv) may comprise identifying the molecular barcode in the cells displaying the phenotype of interest, thereby identifying the effector proteins present in the selected cells. After step (ii), tagging of potential effector proteins may be confirmed using high-throughput imaging and/or in-situ barcode sequencing.

In the methods, step (ii) may comprise integrating an exogenous DNA sequence into an intronic genomic sequence in various cells of the population, wherein the intronic genomic sequence is unique for each of the cells, wherein said integrating is achieved by delivering to the cell population (a) a library of sgRNAs designed to target introns of the effector genes; and (b) a composition comprising (i) an endonuclease-encoding nucleic acid sequence, (ii) a donor plasmid comprising nucleic acid comprising, from 5′ to 3′, a first sgRNA binding site, a splice acceptor site, a sequence encoding the orthogonal ligand binding domain, a splice donor site, and a second sgRNA binding site, and (iii) a donor plasmid-specific gRNA-encoding sequence. The endonuclease may be a Cas endonuclease. The Cas endonuclease may be a Cas9 endonuclease. The composition may further comprise a site-specific guide RNA (gRNA)-encoding nucleic acid sequence. The site-specific and/or donor plasmid-specific guide RNA may be a single gRNA. The site-specific and/or donor plasmid-specific guide RNA may be a CRISPR-RNA (crRNA). The site-specific and/or donor plasmid-specific guide RNA may comprise a fusion of a crRNA and a trans-activating CRISPR RNA (tracrRNA). The guide RNA may comprise a crRNA and a tracrRNA. The endonuclease and the donor plasmid-specific gRNA may be encoded on a single nucleic acid molecule. The endonuclease, the donor plasmid-specific gRNA, and the donor plasmid may be encoded on a single nucleic acid molecule. The endonuclease-encoding nucleic acid sequence, the donor plasmid, the site-specific guide RNA (gRNA)-encoding nucleic acid sequence, and the donor plasmid-specific gRNA-encoding sequence may be present on separate nucleic acid molecules. The first and second sgRNA binding sites of the donor plasmid may comprise the same nucleotide sequence. The donor plasmid may further comprise an antibiotic resistance gene positioned between the splice donor site and the second sgRNA binding site or between the first sgRNA binding site and the splice acceptor site. The antibiotic resistance gene may be blasticidin. The methods may comprise using a library of sgRNAs that target all introns of the effector genes. The methods may comprise using a library of sgRNAs that correspond to all three coding frames. The portion of the donor plasmid comprising the splice acceptor site, the sequence encoding the orthogonal ligand binding domain, and the splice donor site may be integrated into the intronic genomic sequence of a target gene. The orthogonal ligand binding domain may be expressed when the target gene is expressed. The methods may further comprise detecting the expression of the antibiotic resistance gene and/or detecting the expression of the orthogonal ligand binding domain.

In the methods, step (ii) may comprise integrating an exogenous DNA sequence into a genomic sequence of a gene in a cell, such that the exogenous DNA sequence will form either an N-terminal or C-terminal tag on a protein encoded by the gene, by delivering to the cell population a composition comprising (a) prime editing enzymes and (b) a library of pegRNAs comprising the orthogonal ligand binding domain.

The target gene may be an endogenous gene. The target gene may be BCR-Abl, CFTR, MYCN, or mHTT. The target gene may be an exogenous fluorescent protein. The exogenous fluorescent protein may be GFP. The effector proteins may be E3 ligases. The effector proteins may be kinases, phosphatases, E3 ligases, chaperones, deubiquitinating enzymes, methyl transferases, demethylases, acetyltransferase, deacetylating enzymes, glycosyltransferases, and dissagregases.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present invention. The invention may be better understood by reference to one or more of these drawings in combination with the detailed description of specific embodiments presented herein.

FIG. 1. Platform overview. Cells expressing a target protein containing or tagged with a ligand binding domain (including but not limited to FKBP^{F36V}) undergo high throughput endogenous protein tagging for the installation of an orthogonal ligand binding domain (such as HaloTag) into a range of potential effector proteins. In Step 1, a generic dimerizer that binds to both ligand binding domains is added to cells, inducing protein-protein interactions between the target and the effectors. In Step 2, the phenotype of interest is measured to identify viable effectors. Phenotypes of interest can include fluorescence for fluorescent targets, cell abundance for growth-affecting targets, target protein expression levels, target protein localization, or any phenotype that can be measured and selected for.

FIGS. 2A-2E Chemically induced dimerization. (FIG. 2A) The heterobifunctional recruitment approach (exemplified by PROTACs) can lead to protein degradation when an E3 ligase is recruited or diverse phenotypes when alternate effectors are recruited. (FIG. 2B) The linker length between the two ligands must be suitable to induce favourable protein-protein contacts between the two proteins. (FIGS. 2C-2E) Structures of exemplary dimerizing compounds: (FIG. 2C) FKBP-HaloTag Dimerizer; (FIG. 2D) BRD4-HaloTag Dimerizer; (FIG. 2E) BCR-Abl-HaloTag Dimerizer.

FIGS. 3A-3G. Pooled intron tagging facilitates the generation of cell pools with endogenous fusions which are non-disruptive and span most cellular compartments. (FIG. 3A) Illustration of the molecular events leading to intron tagging. (FIG. 3B) Illustration of the experimental pipeline for the generation of pooled tagged cell libraries using intron tagging. (FIG. 3C) Example microscopy images of a pooled tagged library, multiple localization patterns can be observed within the same frame corresponding to a different tagged protein in each cell. (FIG. 3D) Histogram of the number of introns tagged per gene in the sorted library. (FIG. 3E) Distribution of cellular sub compartments in the designed and sorted libraries. (FIG. 3F) Dot plot comparing gene knockout scores with changes in tagged cell frequency over time. (FIG. 3G) Distribution of surface accessibility for tag location within the protein for designed and sorted libraries.

FIGS. 4A-4D. Developing pooled tagging using prime editing. (FIG. 4A) Illustration of tag knockin using prime editing. (FIG. 4B) HEK293T Cells with C-terminal tagging of H2B with split GFP using prime editing. (FIG. 4C) Proportion of fluorescent cells over time after low MOI transduction of prime editing components. PE2 corresponds to tagging without the nicking sgRNA. (FIG. 4D) Cloning strategy for making pooled PE2 tagging plasmid library.

FIGS. 5A-5D. High throughput microscopy and in-situ sequencing for analyzing localization patterns and cellular phenotypes at scale. (FIG. 5A) Images of a pooled tagged library showing both localization of tagged proteins and a single in-situ sequencing cycle. (FIG. 5B) Comparison of bulk amplicon-based sequencing with in-situ sequencing of the same pooled cell library. (FIG. 5C) Illustration of microscopy pipeline. (FIG. 5D) Example images of tagged protein with two introns each corresponding to two distinct tagging locations on the protein.

FIG. 6. exogenous expression of MYCN or CMYC in SHEP cells results in higher proliferation rates.

FIGS. 7A-7D. Leveraging the F-Map Platform Against CFTR and mHTT. (FIG. 7A) Microscopy assay for CFTR membrane proximity [Adapted from Prins et. al., J.B.C., 2020, 16529]. (FIG. 7B) Structure of Lumacaftor derived probe for use in F-Map. (FIG. 7C) Microscopy images of mHTT N-terminal fragments aggregating in iPSC derived Ngn2 neurons. mHTT is expressed w/and w/o an NLS forming aggregates in the nucleus and the cytoplasm respectively. (FIG. 7D) PulSA FACS plot showing the ability to physically separate neurons w/and w/o aggregates.

FIGS. 8A-8C. Generating a model target protein. A) Confocal image and overview of a model target protein in HAP1 cells. A GFP (in our case mClover3) is fused with a ligand binding domain (FKBPF36V) and uniformly expressed throughout the cytoplasm. The susceptibility of the target to degradation is tested with dTAGv-1, which binds to both the FKBP domain and the VHL E3 ligase (PMID 32948771). B) Flow cytometry a clonal fluorescent cell line (pictured in A) treated with 1 μM dTAGv-1 for 22 hours. C) Dose response of dTAGv-1 after 23 hours of treatment.

FIGS. 9A-9C. Generating a tagged library of endogenous effector proteins. A) A targeted library of effectors was designed to have members of the ubiquitin proteasome system (UPS), the autophagy-lysosome pathway, and the cellular chaperone system. Future libraries will be proteome-wide to widen the net for potential effectors of various phenotypes. B) Endogenous proteins were tagged at scale with HaloTag in HAP1s such that each cell has a single protein tagged as described previously (PMID 31239279). Bulk sequencing of the multiplexed cell library confirmed that 90.8% of the 1306 targeted proteins were tagged, with many proteins tagged at more than one site. C) Confocal imaging of the multiplex tag cell library with a magnified inset. Tagged effectors with various localization patterns are shown in white, the model fluorescent target is in cyan, and nuclei are in blue.

FIGS. 10A-10D. Testing a toolbox of generic ligands. A) Schematic of generic ligands targeting both FKBPF36V and HaloTag of various linker lengths. B) Ligands with various linker lengths were used to treat HEK 293 cells expressing both EGFP-FKBPF 36V and STRCP-HT (PMID 28767222) for 23 hours. 0.5 μM dTAGv-1 was used as a positive control. Ligands with longer linkers are more potent in this model. C) 23 hour dose response of GMB-039 and PhosTAC7 (PMID 34780684) in various cell lines. Cell lines include HEK 293s with EGFP-FKBPF36V and βTRCP-HT or HT-PRKN, and HAP1s with mClover3-FKBPF36V and βTRCP-HT or mRuby3-HT7-PRKN. D) Schematic of the HAP1 line expressing mClover3-FKBPF36V and mRuby3-HT7-PRKN for FRET analysis in response to various doses of the generic ligands. Longer ligands induce much stronger FRET than shorter ligands.

FIG. 11. Overview of approach to screen for effectors of degradation. In Step 1, a multiplexed tag library expressing a fluorescent target is generated as described in FIGS. 2 & 3. In Step 2, a generic heterobifunctional ligand with an optimized linker length is used to treat cells. Controls can include vehicle (DMSO), a des-chloro derivative of the ligand, or a ligand with a very short linker length (GMB-017). In Step 3, cells are sorted for the top and bottom 15% of cells by FITC intensity and deep sequenced in Step 4. Finally, in Step 5, putative effectors are validated in various conditions.

FIGS. 12A-12B. Raw data from initial screen. A multiplexed tag library in HAP1 cells was treated with 20 nM PhosTAC7 or the GMB-017 control compound for 24 hours. Each point represents a tagging guide, and in A) the fold change (FC) enrichment of guides in the low fluorescence population is measured for both ligands. Subfigure B) represents the FC difference between both ligands as a function of guide abundance in the PhosTAC7 condition. Tagging guides highlighted in red were among those chosen for validation experiments.

FIGS. 13A-13C. Validation of a hit from the screen by flow cytometry. WDR26 was tagged in HAP1 cells expressing mClover3-FKBPF36V and tested for its ability to degrade the target. A-C) Guides “B7” or “B8” were used to tag endogenous WDR26 and A, B) polyclonal or C) monoclonal cell lines were isolated. Cell lines were labeled with 20 nM HaloTag-TMR to assess their tag homogeneity. Cells were treated with DMSO, PhosTAC7 (PT7), or 1 μM dTAGv-1 and flow plots are overlaid. The degree of target degradation largely depends on the expression level of the effector.

FIG. 14. Annotation of MA plot from FIG. 12B showing WDR26 and other components of the GID/CTLH E3 ligase complex that are likely to also validate as degradation effectors.

FIGS. 15A-15B. Degradation of endogenous BRD4 with WDR26. A) The top schematic represents the ternary complex configuration in the four cell lines tested. “EOI clone A” and “B” lines represent HAP1 cells with endogenously tagged WDR26, the Effector of Interest (EOI). A cell line with a different endogenously tagged protein that is inactive as a degrader is also tested. Finally, the parental cell line without endogenous tags is also used. All cell lines are treated with vehicle, 20 nM PhosTAC7, and increasing concentrations of TM015 (“HT-BRD4 lig”), a chloroalkane-JQ1 heterobifunctional ligand that both binds HaloTag and endogenous BRD4. BRD4 is potently degraded in cells where WDR26 is used as an effector. B) Chemical structure of TM015.

### DETAILED DESCRIPTION

Protein manipulation based on pharmacological induction of protein-protein interactions has immense therapeutic implications. The induced proximity of a targeted protein with a specific effector can induce the target's degradation, stabilization, localization, or a change in its post-translational modifications, which can in turn affect structure and function. This technology has most readily been translated to the field of oncology with small molecules that can induce degradation of oncogenes by inducing proximity with members of the ubiquitin-proteasome system. Unfortunately, very few proteins have been characterized as viable effectors of degradation and even fewer have made their way into clinical trials. Effectors of other forms of protein manipulation, such as stabilization or disaggregation, are rarer still or completely undiscovered.

Here, platforms for the high-throughput discovery of endogenous effectors for any phenotype of interest are provided. Protein-protein interactions are induced between a target of interest and any other protein using a generic heterobifunctional ligand that binds to domains introduced by pooled protein tagging. These platforms, and the subsequent discoveries made by their use, dramatically enhance the ability to leverage targeted protein degradation strategies therapeutically.

For example, the Lig-Map platform enables the selection of suitable E3 ligases for PROTAC applications in a context-dependent fashion. Not all E3 ligases are expressed universally, thus it may be possible to select for E3 ligases that are amenable to the PROTAC approach in one cell type whilst sparing the target protein in another using dual cell-line screening via the Lig-Map platform. In addition, the Lig-Map platform can readily be ported between cell lines since it uses endogenous tagging strategies rather than exogenous protein expression. Additionally, recent reports have demonstrated that resistance mechanisms to PROTACs can arise via the down-regulation of E3 ligase expression. The Lig-Map platform, in concert with CRISPR knockout screens, enables the discovery of essential ligases that are amenable to the PROTAC approach and therefore circumnavigate this resistance concern.

Additionally, the F-Map platform enables the unbiased discovery of effector recruitment strategies, abrogating the need for extensive medicinal chemistry to provide proof of principle, and therefore speeding up the discovery of much needed novel therapeutic mechanisms. Current approaches rely on hypothesis generation and extensive method development prior to proof of principle, whereas the F-Map approach removes the requirement for this expensive and time-consuming process, as well as systematically testing every possible approach in a rapid and concise workflow. Beyond the cancer, neurodegenerative disease, and progressive, genetic disease examples given here, the F-map approach is amenable to essentially any human disease.

### I. CHEMICALLY INDUCED DIMERIZATION

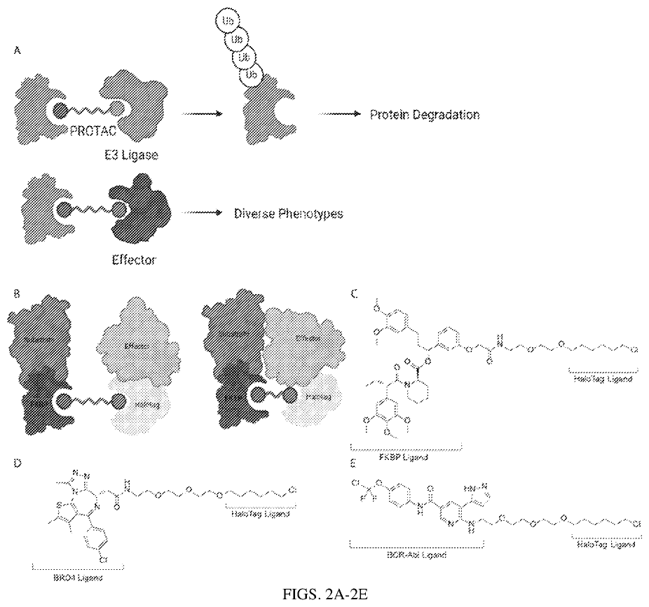

Chemically induced dimerization forms the basis of the PROTAC technology and can conceivably be applied to other effector domains (FIG. 2A). Crucially for successful ternary complex formation are additive protein-protein contacts hence the requirement for linker optimization (FIG. 2B). The platforms provided herein rely on the use of heterobifunctional compounds that recognize bio-orthogonal protein tags on either end. For example, on one end a chloroalkane may be used to covalently label a HaloTag fusion protein, and on the other a bump-hole mutant FKBP_{F36V} (also referred to herein as FKBP) (FIG. 2C). This approach has previously been used to induce ternary complex formation between an E3 ligase and substrate which forms the feasibility basis for our proposal. However, the platforms provided herein enable this to be performed in a multiplexed, proteome-wide fashion and to use endogenous proteins, rather than overexpressed proteins, which are known to result in false positives and complicate analysis in induced proximity studies.

### II. GENERATION OF CELL POOLS WITH ENDOGENOUSLY TAGGED EFFECTOR PROTEINS

Two approaches are provided for the generation of cell pools with tagged endogenous proteins. Pooled intron tagging, for adding internal tags, and pooled prime-editing tagging, for adding C- and N-terminal tags. As the generation of pools of tagged cells forms the basis for this proposal, we provide here preliminary data to support the feasibility of this proposal.

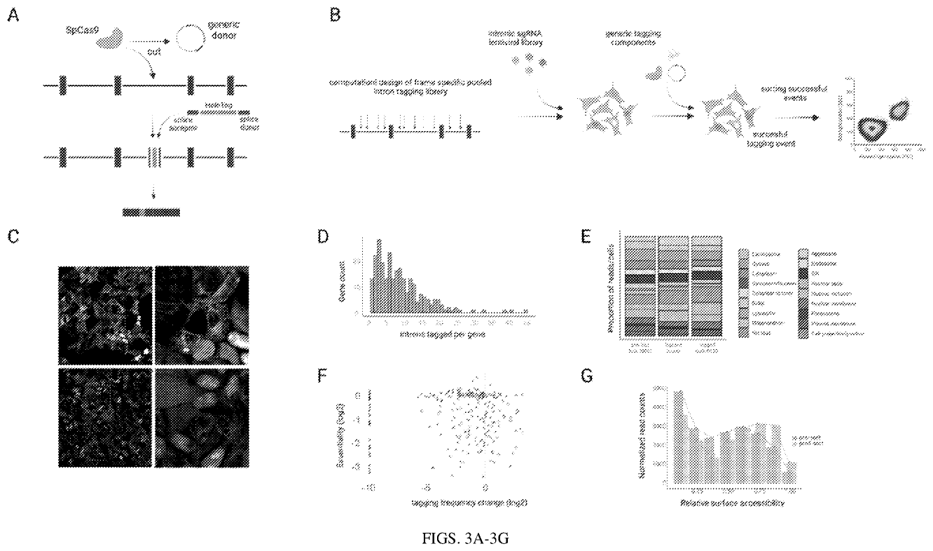

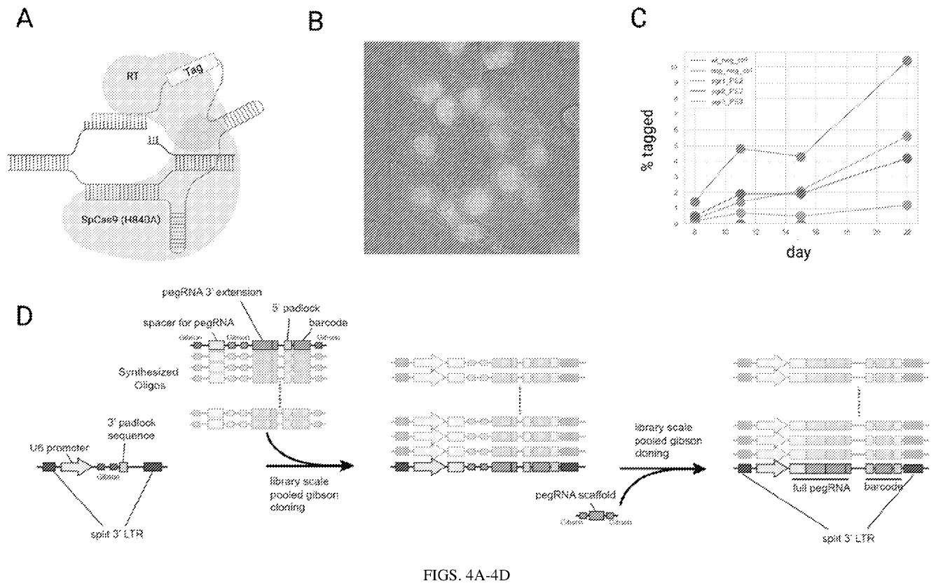

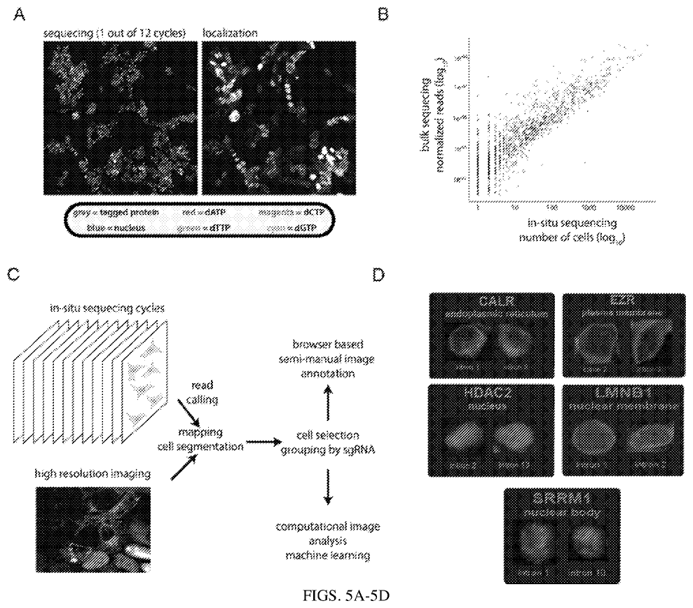

Pooled intron tagging: Pooled gene tagging facilitated by the formation of double strand breaks (DSB) requires the use of a generic donor, as there are currently no scalable ways for pooled delivery of gene-specific homology-based donors concomitantly with correct sgRNA. To facilitate pooled tagging with a generic donor, intron tagging, an approach that adds a synthetic exon within introns of the target genes for tagging using non homologous end joining (NHEJ), is used. Cas9 and an sgRNA are delivered to generate a DSB within an intron of interest together with the delivery of a generic plasmid donor (no homology arms to the target), which is also cut by Cas9 in cells to generate a linearized donor (FIG. 3A). To increase apparent tagging efficiency, an antibody selection cassette, which is in the donor, but outside the splice acceptor and donor sites, so it is added to the genome but not to the tagged protein, is used. To choose sgRNAs for such a library, a computational algorithm that considers all transcripts of a specific gene and sgRNA on and off target score may be used. The algorithm may use a greedy approach to choose effective sgRNAs (using on/off target scores) while trying to uniformly place them across the intron. The feasibility of this approach was tested, as described in Example 1.

An exemplary protocol for generating and isolating intron tagged cell clones by intron targeting is provided below.

### Materials and Reagents

HEK293 cells (ATCC CRL-1573)
   Can also work with (but not limited to) Hela cells, H9 hESCs, or HAP1 cells.
Cell culture reagents (Thermo Fischer Scientific): DMEM, trypsin, Opti-MEM
Transfection-grade PEI (Polysciences, cat. #24765)
Plasmid encoding ligand binding domain generic donor
Plasmid encoding target sgRNA
   The appropriate spacer sequences can be cloned into the lentiGuide-Puro plasmid (Addgene #52963). sgRNA sequences can be chosen using the sgRNA designer provided by the Broad institute (available at portals. broadinstitute. org/gpp/public/analysis-tools/sgrna-design).
Plasmid encoding sgRNA against donor
Plasmid encoding Cas9
   Can be used separately, such as on the lentiCas9-Blast plasmid (Addgene #53962), or included on the sgRNA plasmids (e.g. Addgene #52961).

### Equipment

Cell sorter (BD FACSAria Fusion or any other similar cell sorter)
Flow cytometry tubes (12×75 mm)
Cell Straining Kit (70 μm)
Cell culture plates (12-well plate, 10 cm dish, 96-well plate)
Cell culture incubator (37° C., 5% CO₂).

### Procedure

Day 1: Plate cells: 1) Plate HEK 293 cells into at least 2 wells of a 12-well plate, such that they will be 60-80% confluent on the following day (160-200×10³ cells/well).
Day 2: Transfect cells: 2) To 100 μl Opti-MEM, add plasmids encoding the generic donor, the donor sgRNA, the target sgRNA, and Cas 9 in a 5:1:1:1 molar ratio, respectively. A total amount of 1360 ng of plasmid DNA is mixed with 4.08 μl PEI (1 μg DNA: 3 μl PEI ratio). 3) An additional reaction with PEI reagent only is used as the “mock” transfection. 4) Vortex and incubate transfection mixtures for at least 15 minutes. 5) Add each mixture dropwise to a single well of a 12-well plate.
Day 3: Wash and replate cells.
Day 7-8: Analyze and sort cells.

Pooled tagging with prime editors: Prime editing (PE) may be used to prepare pooled libraries in which the C- and N-termini of each gene is tagged. Terminal tagging is frequently used for gene tagging as it is perceived to be less likely to disrupt the tagged protein structure and function. An additional advantage of PE is that the donor is encoded as an extension of the sgRNA (i.e., pegRNA) which can be delivered by lentiviral transduction increasing the cell models that are amenable for pooled tagging (FIG. 4A). One challenge in using this approach is library design and cloning in a way that will (a) enable readout of the sgRNA sequence using in-situ sequencing and (b) avoid template switching in library construction, lentiviral production and readout. To address this, a two-step, large-scale, cloning protocol to integrate each pegRNA with a unique barcode into a CROPseq backbone vector has been developed. In addition, testing for template switching and lentiviral recombination at multiple steps may be performed (FIG. 4D). Another limitation of PE tagging is the size of the tag for insertion, which is much more restricted than intron tagging. In order to adapt this approach to scalable recruitment, a split HaloTag, which was recently developed, may be used as a donor tag.

Characterization and validation of pooled tagged libraries: Various methods may be used to ensure nondisruptive gene tagging of the effector proteins in cell libraries. First, in the process of library generation, a TMR HaloTag ligand may be added in order to sort out cells with a fluorescent signal. In many cases, tagging that disrupts the folding of the fused protein results in a diffused localization pattern and very low fluorescent signal. Thus, FACS based sorting of only cells with medium to high expression levels may eliminate a large proportion of disruptive tagging events.

Next, as many proteins are essential for cell growth, bulk next generation sequencing of the sorted library at two time points may be used to mark sgRNAs that are depleted out of the library over time. In the pilot library described in Example 1, knockout depletion (by running a small CRISPR knockout screen) was compared with changes in frequency of tagging sgRNA in the sorted libraries, and no correlation between the two was found (FIG. 3F), suggesting that intron tagging is able to select for non-disruptive gene fusions. Additional support for this comes from analysis of selected introns in the tagged proteins, in which a large increase in introns positioning the tag in an exposed location within the protein were found (FIG. 3G).

Finally, automated high throughput image analysis followed by in-situ sgRNA (or barcode in the case of PE libraries) sequencing may be used to identify the tagged protein in each cell (FIG. 5A). First, the TMR HaloTag ligand is added to the cell, so tagged proteins can be visualized, then the cells are fixed for high throughput phenotyping of localization patterns. Cells are imaged at high resolution using a 40× objective together with a nuclear and cytoplasmic dyes using a motorized programmable microscope stage, to cover multiple wells while recording the physical location of each image frame. At the next step, in-situ sequencing is performed following a protocol for pooled imaging based CRISPR screens. Briefly, in this approach, the sgRNA sequence is reverse transcribed in-situ followed by padlock cyclic amplification and sequencing-by-synthesis (SBS) using Illumina reagents and cycles of automated microscope imaging to read an individual nucleotide in each imaging cycle at a 10× resolution. Running twelve imaging cycles results in identification of the twelve first nucleotides of each sgRNA/barcode, which is sufficient to uniquely match the sgRNA in each cell to the transduced library. Using this approach, in-situ sequencing corresponds well with standard PCR-based bulk sequencing of the sgRNA pools (FIG. 5B), and the sequencing reads can be mapped to localization patterns and cells computationally selected with the same sgRNAs for image arrays to examine the localization patterns. Data from such experiments may be analyzed both semi-manually and using machine learning approaches to ensure proper localization of the tagged proteins (FIG. 5C). Moreover, sgRNAs may be selected such that more than one intron is tagged with similar localization patterns confirming that localization is due to the protein and not the tag (FIG. 5D). However, proper localization is not required for the present approach to be successful, only for the tagged protein to be folded properly. The use of pooled image analysis and in-situ sequencing will also be used for expanding the ways in which the effect of recruitment on the target protein may be measured. For example, by measuring target protein degradation, localization and additional imaging-based cellular phenotypes.

### III. IDENTIFICATION OF DEGRADATION COMPETENT E3 LIGASES

There are numerous examples of successful PROTAC development campaigns with several PROTAC molecules currently in clinical trials for a range of oncology indications. Whilst the promise of the PROTAC technology is beginning to be realized, there are still several hurdles in the development of the PROTAC strategy. The largest challenge is the incomplete understanding of the E3 ligases in the human proteome which are amenable to high jacking via PROTACs. To date, only 7 of the postulated 800+E3 ligases in the human genome have been utilized in PROTACs and of the disclosed candidates in clinical trials, all of them use the same E3 ligase, CRBN9. As the importance of surface complementarity between the ligase and the neo-substrate has emerged, is has become clear that there is an urgent need to expand the toolbox of E3 ligases which can be recruited via PROTACs to ensure that a stable ternary complex can be identified for any given protein. Small scale efforts have been applied to expand the E3 ligase armory for PROTAC development by unbiased or serendipitous ligand discovery for E3 ligases and then conversion into PROTACs or by tag-based approaches to probe the utility of certain E3 ligases for PROTAC recruitment. Whilst these approaches have proven moderately successful, they have failed to deliver a road map of E3 ligase suitability for PROTAC development. Global profiling of E3 ligase “PROTACability” will be crucial for the success of the targeted protein degradation paradigm in the future, not least because it will enable the prioritization of ligand development for the recruitment for the most promising E3 ligases.

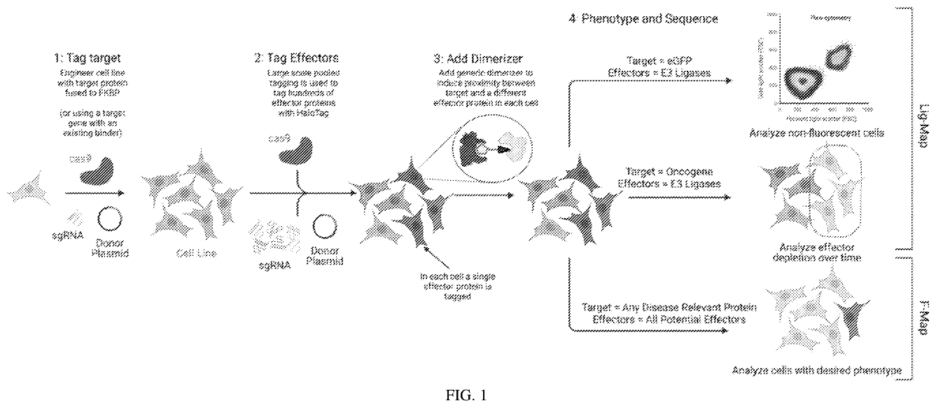

Provided herein is an E3 ligase-wide profiling technology based on endogenous tagging of every postulated E3 ligase with bio-orthogonal tags which can be used to probe the recruitability of each ligase to a model substrate in a multiplexed fashion. This approach, called Ligase Mapping (Lig-Map) (FIG. 1, see Example 3), enables the generation of a global profile of degradation competent E3 ligases, across multiple cellular localizations. This platform outputs the potential effectors/target pair and information about the potential optimal linker length and effector/target configuration/orientation, which provides a starting point for subsequent med chem development of small molecules that will bring that specific effector/target pair together.

### IV. IDENTIFICATION OF A SUITABLE E3 LIGASE FOR A TARGET

Another significant hurdle in the successful development of a PROTAC, is the identification of a suitable E3 ligase for the target of interest. Currently this is done empirically given that only 3-4 ligases are routinely utilized; however this is inefficient, requiring extensive synthetic and medicinal chemistry, and often fails to identify a feasible ligase. Even if each of the postulated 800 human E3 ligases were amenable to the PROTAC approach and a small molecule ligand was available for each one, the identification of the optimal ligase for a protein of interest would still be a gargantuan challenge. Given that these ligands are not available, and the significant medicinal chemistry resource required to develop them, it is even more important to know which ligase is the most suitable for the target of interest. This knowledge would significantly speed up the development of therapeutic strategies for multiple cancers and other disease types. Using the Lig-Map approach (FIG. 1), in a different mode, the optimal E3 ligase for any given disease relevant protein can be identified rapidly using either a dual tagging strategy or a ligand-tag strategy.

The Lig-Map approach can be employed to rapidly identify promising E3 ligases for therapeutically relevant proteins across proteins and cell models (see Example 4). To exemplify this approach, two targets will be used, one which has been validated as a PROTAC target previously and one with therapeutic potential but no reported PROTAC.

### V. IDENTIFICATION OF ADDITIONAL EFFECTOR PROTEINS

Beyond the recruitment of E3 ligases, the power of induced proximity has already begun to be realized with technologies reported for the recruitment of kinases, phosphatases, autophagy modulators, and acetyltransferases, but this exploration of potential effector proteins has been performed in a somewhat random fashion based on available chemical tools and not based on fundamental applicability or generalizability of effector proteins. To rationally explore the ability of proteins to be recruited to one another for therapeutic benefit, the Lig-Map technology will be expanded to a much larger selection of potential effector proteins resulting in the F-Map (FIG. 1) platform. To demonstrate the power of this approach, the F-Map technology will be applied to two differential disease relevant examples. First, proteins that can stabilize CFTR, a protein prematurely degraded in cystic fibrosis, will be profiled, since a recent report demonstrated the recruitment of a deubiquitinating enzyme can stabilize it with therapeutic benefit. Second, the F-Map technology will be used to explore proteins that may induce the diffusion of aggregated Huntingtin (HTT) inclusion bodies, present in patients with Huntington's Disease.

Inducing proximity as a therapeutic strategy has a great deal of potential application beyond E3 ligase mediated targeted protein degradation. The Lig-Map platform will be expanded to systematically cover other potential effector proteins (denoted Effector Mapping (or F-Map) Technology; see Example 5) by expanding our tagging strategy to all enzymatically active proteins, including, but not limited to, kinases, phosphatases, E3 ligases, chaperones, deubiquitinating enzymes, methyl transferases, demethylases, acetyltransferase, deacetylating enzymes, glycosyltransferases, and dissagregases. Several recent reports have demonstrated that other PTM modifying enzymes are applicable to induced proximity approaches, but the F-Map platform will enable the systematic and proteome wide assessment of protein-protein induction technologies.

Beyond the two exemplar targets described in Example 5, the F-Map platform may be readily applied to any disease relevant target with a measurable phenotype such that an induced proximity approach could readily be identified, thus increasing the speed and scope of drug discovery efforts. Currently, one of the major reasons that drug discovery campaigns fail, is misunderstood biology of the target. With the use of the F-Map platform, the therapeutic approach is validated during the discovery thus circumnavigating this concern.

### VI. CRISPR/CAS SYSTEMS

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

The CRISPR/Cas nuclease or CRISPR/Cas nuclease system can include a non-coding RNA molecule (guide) RNA, which sequence-specifically binds to DNA, and a Cas protein (e.g., Cas9), with nuclease functionality (e.g., two nuclease domains). One or more elements of a CRISPR system can derive from a type I, type II, or type III CRISPR system, e.g., derived from a particular organism comprising an endogenous CRISPR system, such as Streptococcus pyogenes.

In some aspects, a Cas nuclease and gRNA (including a fusion of crRNA specific for the target sequence and fixed tracrRNA) are introduced into the cell. In general, target sites at the 5′ end of the gRNA target the Cas nuclease to the target site, e.g., the gene, using complementary base pairing. The target site may be selected based on its location immediately 5′ of a protospacer adjacent motif (PAM) sequence, such as typically NGG, or NAG. In this respect, the gRNA is targeted to the desired sequence by modifying the first 20, 19, 18, 17, 16, 15, 14, 14, 12, 11, or 10 nucleotides of the guide RNA to correspond to the target DNA sequence. In general, a CRISPR system is characterized by elements that promote the formation of a CRISPR complex at the site of a target sequence. Typically, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex.

The CRISPR system can induce double stranded breaks (DSBs) at the target site, followed by disruptions as discussed herein. In other embodiments, Cas9 variants, deemed “nickases,” are used to nick a single strand at the target site. Paired nickases can be used, e.g., to improve specificity, each directed by a pair of different gRNAs targeting sequences such that upon introduction of the nicks simultaneously, a 5′ overhang is introduced. In other embodiments, catalytically inactive Cas9 is fused to a heterologous effector domain such as a transcriptional repressor or activator, to affect gene expression.

The target sequence may comprise any polynucleotide, such as DNA or RNA polynucleotides. The target sequence may be located in the nucleus or cytoplasm of the cell, such as within an organelle of the cell. Generally, a sequence or template that may be used for recombination into the targeted locus comprising the target sequences is referred to as an “editing template” or “editing polynucleotide” or “editing sequence”. In some aspects, an exogenous template polynucleotide may be referred to as an editing template. In some aspects, the recombination is homologous recombination.

Typically, in the context of an endogenous CRISPR system, formation of the CRISPR complex (comprising the guide sequence hybridized to the target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. The tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of the CRISPR complex, such as by hybridization along at least a portion of the tracr sequence to all or a portion of a tracr mate sequence that is operably linked to the guide sequence. The tracr sequence has sufficient complementarity to a tracr mate sequence to hybridize and participate in formation of the CRISPR complex, such as at least 50%, 60%, 70%, 80%, 90%, 95% or 99% of sequence complementarity along the length of the tracr mate sequence when optimally aligned.

One or more vectors driving expression of one or more elements of the CRISPR system can be introduced into the cell such that expression of the elements of the CRISPR system direct formation of the CRISPR complex at one or more target sites. Components can also be delivered to cells as proteins and/or RNA. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. The vector may comprise one or more insertion sites, such as a restriction endonuclease recognition sequence (also referred to as a “cloning site”). In some embodiments, one or more insertion sites are located upstream and/or downstream of one or more sequence elements of one or more vectors. When multiple different guide sequences are used, a single expression construct may be used to target CRISPR activity to multiple different, corresponding target sequences within a cell.

A vector may comprise a regulatory element operably linked to an enzyme-coding sequence encoding the CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10,Csx16, CsaX, Csx3, Csx1, Csx15, Csf1, Csf2, Csf3, Csf4, homologs thereof, or modified versions thereof. These enzymes are known; for example, the amino acid sequence of S. pyogenes Cas9 protein may be found in the SwissProt database under accession number Q99ZW2.

The CRISPR enzyme can be Cas9 (e.g., from S. pyogenes or S. pneumonia). The CRISPR enzyme can direct cleavage of one or both strands at the location of a target sequence, such as within the target sequence and/or within the complement of the target sequence. The vector can encode a CRISPR enzyme that is mutated with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). In some embodiments, a Cas9 nickase may be used in combination with guide sequence(s), e.g., two guide sequences, which target respectively sense and antisense strands of the DNA target. This combination allows both strands to be nicked and used to induce NHEJ or HDR.

In some embodiments, an enzyme coding sequence encoding the CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

In general, a guide sequence is any polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more.

Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), Clustal W, Clustal X, BLAT, Novoalign (Novocraft Technologies, ELAND (Illumina, San Diego, Calif.), SOAP (available at soap. genomics. org. cn), and Maq (available at maq.sourceforge.net).

The CRISPR enzyme may be part of a fusion protein comprising one or more heterologous protein domains. A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A CRISPR enzyme may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4A DNA binding domain fusions, and herpes simplex virus (HSV) BP16 protein fusions. Additional domains that may form part of a fusion protein comprising a CRISPR enzyme are described in US 20110059502, incorporated herein by reference.

Compositions provided herein comprise targeting constructs comprising at least one targeting sequence. In some embodiments, the targeting construct comprises at least two targeting sequences. Targeting sequences herein are nucleic acid sequences recognized and cleaved by a nuclease disclosed herein in a sequence specific manner. In some embodiments, the targeting sequence is about 9 to about 12 nucleotides in length, from about 12 to about 18 nucleotides in length, from about 18 to about 21 nucleotides in length, from about 21 to about 40 nucleotides in length, from about 40 to about 80 nucleotides in length, or any combination of subranges (e.g., 9-18, 9-21, 9-40, and 9-80 nucleotides). In some embodiments, the targeting sequence comprises a nuclease binding site. In some embodiments the targeting sequence comprises a nick/cleavage site. In some embodiments, the targeting sequence comprises a protospacer adjacent motif (PAM) sequence.

In some embodiments, the target nucleic acid sequence (e.g., protospacer) is 20 nucleotides. In some embodiments, the target nucleic acid is less than 20 nucleotides. In some embodiments, the target nucleic acid is at least 5, 10, 15, 16, 17, 18, 19, 20, 21, 22, 23, 24, 25, 30 or more nucleotides. The target nucleic acid, in some embodiments, is at most 5, 10, 15, 16, 17, 18, 19, 20, 21, 22, 23, 24, 25, 30 or more nucleotides. In some embodiments, the target nucleic acid sequence is 16, 17, 18, 19, 20, 21, 22, or 23 bases immediately 5′ of the first nucleotide of the PAM. In some embodiments, the target nucleic acid sequence is 16, 17, 18, 19, 20, 21, 22, or 23 bases immediately 3′ of the last nucleotide of the PAM. In some embodiments, the target nucleic acid sequence is 20 bases immediately 5′ of the first nucleotide of the PAM. In some embodiments, the target nucleic acid sequence is 20 bases immediately 3′ of the last nucleotide of the PAM. In some embodiments, the target nucleic acid sequence is 5′ or 3′ of the PAM.

A targeting sequence, in some embodiments includes nucleic acid sequences present in a target nucleic acid to which a nucleic acid-targeting segment of a complementary strand nucleic acid binds. For example, targeting sequences, in some embodiments, include sequences to which a complementary strand nucleic acid is designed to have base pairing. A targeting sequence in some embodiments comprises any polynucleotide, which is located, for example, in the nucleus or cytoplasm of a cell or within an organelle of a cell, such as a mitochondrion or chloroplast. Targeting sequences include cleavage sites for nucleases. A targeting sequence, in some embodiments, is adjacent to cleavage sites for nucleases.

The nuclease cleaves the nucleic acid, in some embodiments, at a site within or outside of the nucleic acid sequence present in the target nucleic acid to which the nucleic acid-targeting sequence of the complementary strand binds. The cleavage site, in some embodiments, includes the position of a nucleic acid at which a nuclease produces a single-strand break or a double-strand break. For example, formation of a nuclease complex comprising a complementary strand nucleic acid hybridized to a protease recognition sequence and complexed with a protease results in cleavage of one or both strands in or near (e.g., within 1, 2, 3, 4, 5, 6, 7, 8, 9, 10, 19, 20, 23, 50, or more base pairs from) the nucleic acid sequence present in a target nucleic acid to which a spacer region of a complementary strand nucleic acid binds. The cleavage site, in some embodiments, is on only one strand or on both strands of a nucleic acid. In some embodiments, cleavage sites are at the same position on both strands of the nucleic acid (producing blunt ends) or are at different sites on each strand (producing staggered ends). Staggered ends, in some embodiments, are 5′ or 3′ overhang sticky-ends. Staggered ends, in some embodiments, are produced by sticky-end producing nucleases (e.g., Cpfl). In some embodiments, staggered ends are produced, for example, by using two nucleases, each of which produces a single-strand break at a different cleavage site on each strand, thereby producing a double-strand break. For example, a first nickase creates a single-strand break on the first strand of double-stranded DNA (dsDNA), and a second nickase creates a single-strand break on the second strand of dsDNA such that overhanging sequences are created. In some cases, the nuclease recognition sequence of the nickase on the first strand is separated from the nuclease recognition sequence of the nickase on the second strand by at least 2, 3, 4, 5, 6, 7, 8, 9, 10, 15, 20, 25, 30, 40, 50, 75, 100, 250, 500, or 1000 base pairs.

Site-specific cleavage of a target nucleic acid by a nuclease, in some embodiments, occurs at locations determined by base-pairing complementarity between the complementary strand nucleic acid and the target nucleic acid. Site-specific cleavage of a target nucleic acid by a nuclease protein, in some embodiments, occurs at locations determined by a short motif, called the protospacer adjacent motif (PAM), in the target nucleic acid. For example, the PAM flanks the nuclease recognition sequence at the 3′ end of the recognition sequence. For example, the cleavage site of the nuclease, in some embodiments, is about 1 to about 25, or about 2 to about 5, or about 19 to about 23 base pairs (e.g., 3 base pairs) upstream or downstream of the PAM sequence. In some embodiments, the cleavage site of the nuclease is 3 base pairs upstream of the PAM sequence. In some embodiments, the cleavage site of the nuclease is 19 bases on the (+) strand and 23 base on the (−) strand, producing a 5′ overhang 5 nucleotides (nt) in length. In some cases, the cleavage produces blunt ends. In some cases, the cleavage produces staggered or sticky ends with 5′ overhangs. In some cases, the cleavage produces staggered or sticky ends with 3′ overhangs.

Orthologs of various nuclease proteins utilize different PAM sequences. For example different Cas proteins, in some embodiments, recognize different PAM sequences. For example, in S. pyogenes, the PAM is a sequence in the target nucleic acid that comprises the sequence 5′-XRR-3′, where R is either A or G, where X is any nucleotide and X is immediately 3′ of the target nucleic acid sequence targeted by the spacer sequence. The PAM sequence of S. pyogenes Cas9(SpyCas9 ) is 5′-XGG-3′, where X is any DNA nucleotide and is immediately 3′ of the nuclease recognition sequence of the non-complementary strand of the target DNA. The PAM of Cpfl is 5′-TTX-3′, where X is any DNA nucleotide and is immediately 5′ of the nuclease recognition sequence.

### VII. METHODS FOR DELIVERY

Any suitable delivery method is contemplated to be used for delivering the compositions of the disclosure to a cell population. The individual components of the HITI system (e.g., nuclease and/or the exogenous DNA sequence), in some embodiments, are delivered simultaneously or temporally separated. The choice of method of genetic modification is dependent on the type of cell being transformed and/or the circumstances under which the transformation is taking place.

In some embodiments, a method as disclosed herein involves contacting a target DNA or introducing into a cell (or a population of cells) one or more nucleic acids comprising nucleotide sequences encoding a complementary strand nucleic acid (e.g., gRNA), a site-directed modifying polypeptide (e.g., Cas protein), and/or a exogenous DNA sequence. Suitable nucleic acids comprising nucleotide sequences encoding a complementary strand nucleic acid and/or a site-directed modifying polypeptide include expression vectors, where an expression vector comprising a nucleotide sequence encoding a complementary strand nucleic acid and/or a site-directed modifying polypeptide is a recombinant expression vector.

Non-limiting examples of delivery methods or transformation include, for example, viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, and nanoparticle-mediated nucleic acid delivery.

In some aspects, the present disclosure provides methods comprising delivering one or more polynucleotides, such as or one or more vectors as described herein, one or more transcripts thereof, and/or one or proteins transcribed therefrom, to a host cell. In some aspects, the disclosure further provides cells produced by such methods, and organisms (such as animals, plants, or fungi) comprising or produced from such cells. In some embodiments, a nuclease protein in combination with, and optionally complexed with, a complementary strand sequence is delivered to a cell. Conventional viral and non-viral based gene transfer methods are contemplated to be used to introduce nucleic acids in mammalian cells or target tissues. Such methods are used to administer nucleic acids encoding components of a HITI system to cells in culture, or in a host organism. Non-viral vector delivery systems include DNA plasmids, RNA (e.g. a transcript of a vector described herein), naked nucleic acid, and nucleic acid complexed with a delivery vehicle, such as a liposome. Viral vector delivery systems can include DNA and RNA viruses, which can have either episomal or integrated genomes after delivery to the cell.

Methods of non-viral delivery of nucleic acids can include lipofection, nucleofection, microinjection, electroporation, 1, biolistics, virosomes, liposomes, immunoliposomes, polycation or lipid: nucleic acid conjugates, naked DNA, artificial virions, and agent-enhanced uptake of DNA.

A host cell is alternatively transiently or non-transiently transfected with one or more vectors described herein. In some embodiments, a cell is transfected as it naturally occurs in a subject. In some embodiments, a cell is taken or derived from a subject and transfected. In some embodiments, a cell is derived from cells taken from a subject, such as a cell line. In some embodiments, a cell transfected with one or more vectors described herein is used to establish a new cell line comprising one or more vector-derived sequences. In some embodiments, a cell transiently transfected with the components of a CRISPR system as described herein (such as by transient transfection of one or more vectors, or transfection with RNA), and modified through the activity of a CRISPR complex, is used to establish a new cell line comprising cells containing the modification but lacking any other exogenous sequence.

In some embodiments, a nucleotide sequence encoding a complementary strand nucleic acid and/or a site-directed modifying polypeptide is operably linked to a control element, e.g., a transcriptional control element, such as a promoter. The transcriptional control element is functional, in some embodiments, in either a eukaryotic cell, e.g., a mammalian cell, or a prokaryotic cell (e.g., bacterial or archaeal cell). In some embodiments, a nucleotide sequence encoding a complementary strand nucleic acid and/or a site-directed modifying polypeptide is operably linked to multiple control elements that allow expression of the nucleotide sequence encoding a complementary strand nucleic acid and/or a site-directed modifying polypeptide in prokaryotic and/or eukaryotic cells. Depending on the host/vector system utilized, any of a number of suitable transcription and translation control elements, including constitutive and inducible promoters, transcription enhancer elements, transcription terminators, etc. may be used in the expression vector (e.g., U6 promoter, HI promoter, etc.).

In some embodiments, a complementary strand nucleic acid and/or a site-directed modifying polypeptide is provided as RNA. In such cases, the complementary strand nucleic acid and/or the RNA encoding the site-directed modifying polypeptide is produced by direct chemical synthesis or may be transcribed in vitro from a DNA encoding the complementary strand nucleic acid. The complementary strand nucleic acid and/or the RNA encoding the site-directed modifying polypeptide are synthesized in vitro using an RNA polymerase enzyme (e.g., T7 polymerase, T3 polymerase, SP6 polymerase, etc.). Once synthesized, the RNA directly contacts a target DNA or is introduced into a cell using any suitable technique for introducing nucleic acids into cells (e.g., microinjection, electroporation, transfection, etc).

### VIII. KITS AND DIAGNOSTICS

In various aspects of the invention, a kit is envisioned containing the necessary components to insert a ligand binding domain sequence in an intron or terminus of one or more target gene. The kit may comprise one or more sealed vials containing any of such components. In some embodiments, the kit may also comprise a suitable container means, which is a container that will not react with components of the kit, such as an eppendorf tube, an assay plate, a syringe, a bottle, or a tube. The container may be made from sterilizable materials such as plastic or glass.

The kit may further include an instruction sheet that outlines the procedural steps of the methods set forth herein and will follow substantially the same procedures as described herein or are known to those of ordinary skill. The instruction information may be in a computer readable media containing machine-readable instructions that, when executed using a computer, cause the display of a real or virtual procedure of transfecting or electroporating a CRISPR system in cells in order to insert a reporter encoding sequence in an intron of one or more target gene.

### IX. DEFINITIONS

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the inherent variation in the method being employed to determine the value, the variation that exists among the study subjects, or a value that is within 10% of a stated value.

### X. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Pooled Intron Tagging

To test the feasibility of using pooled intron tagging for the generation of a pool of tagged cells, an sgRNA library spanning the introns of 266 effector genes chosen to span a wide range of localization patterns was designed. The sgRNAs were chosen using a computational algorithm that considers all transcripts of a specific gene and sgRNA on and off target score. The algorithm used a greedy approach to choose effective sgRNAs (using on/off target scores) while trying to uniformly place them across the intron. This pilot library contained 21,966 sgRNAs designed to target all introns of the effector genes with 10 sgRNAs each, split into three sub-libraries corresponding to the three coding frames. The sgRNAs in the library were cloned into a CROPSeq vector, which allows for sgRNA identification with in-situ sequencing. The lentiviral sgRNA library was delivered into HAP1 cells at a low MOI (following standard CRISPR screening protocols) and selected with puromycin. Transduced cells were then expanded and transfected with Cas9, a generic donor designed to add a HaloTag flanked by flexible linkers, and a sgRNA designed to linearize the donor. The library was divided into three separate sub libraries according to the three coding frames and remixed after selection. A TMR HaloTag ligand was added to enable the sorting of cells with tagged endogenous proteins (FIGS. 3B,3C). Sorted cell pools were analyzed by both bulk sequencing and high-throughput microscopy coupled with in-situ sequencing (FIGS. 5A-5C).

In this test library, 230 genes were tagged (>85% success rate), most with more than one intron, corresponding to multiple tagging sites per gene (FIG. 3D), providing internal redundancy to account for tag effects on protein localization and function. Effector proteins with a wide range of cellular compartments were selected, such that the ability to tag proteins with very diverse localization patterns would be tested. Successful tagging and similar success rates were seen in all compartments (FIG. 3E). To show that using intron tagging can select for non-disruptive protein fusions, proteins that are highly essential for cellular growth when disrupted were included in the library. No depletion of these proteins in the sorted and expanded library was found, in contrast to a CRISPR-knockout screen targeting these genes, indicating that intron tagging did not disrupt their growth-related function (FIG. 3F). Protein surface accessibility was also analyzed, and successful tagging preferentially occurred in exposed areas of the protein (FIG. 3G). The last step in validating non-disruptive tagging was the comparison of localization patterns of the same protein using different introns, which was done using microscopy (FIG. 5D).

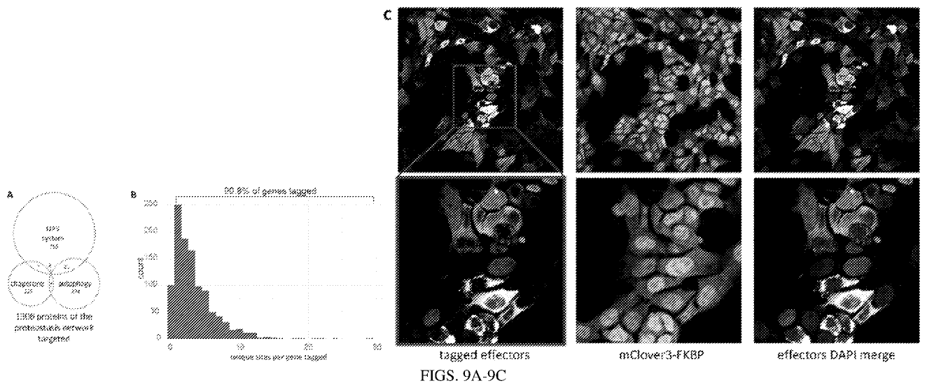

In a further test, a tagged library of targeted endogenous effector proteins was generated (FIGS. 9A-9C). The targeted library of effectors was designed to have members of the ubiquitin proteasome system (UPS), the autophagy-lysosome pathway, and the cellular chaperone system (FIG. 9A). Future libraries will be proteome-wide to widen the net for potential effectors of various phenotypes. Endogenous proteins were tagged at scale with HaloTag in HAP1s such that each cell has a single protein tagged as described previously (PMID 31239279). Bulk sequencing of the multiplexed cell library confirmed that 90.8% of the 1306 targeted proteins were tagged, with many proteins tagged at more than one site (FIG. 9B). FIG. 9C provides confocal imaging of the tagged cell line with a magnified inset. Tagged effectors with various localization patterns are shown in white, the model fluorescent target is in cyan, and nuclei are in blue.

### Example 2—Pooled Tagging With Prime Editors

As a first step to establishing PE as a technique for pooled gene tagging, low MOI transduction of pegRNAs into cell lines previously engineered to have the PE enzymes integrated in them (using piggybac transposase) was shown to result in efficient tagging which also increases over time (FIGS. 4B,4C). One challenge in using this approach is library design and cloning in a way that will (a) enable readout of the sgRNA sequence using in-situ sequencing and (b) avoid template switching in library construction, lentiviral production and readout. To overcome these challenges, a two-step, large-scale, cloning protocol has been designed to integrate each pegRNA with a unique barcode into a CROPseq backbone vector. Testing for template switching and lentiviral recombination will be performed at multiple steps (FIG. 4D). To test the feasibility of pooled PE tagging, a library against 60 proteins was designed using different linker and homology lengths with the aim of identifying parameters that increase the rate of PE tagging. Following library construction, the tagged cells will be characterized using high-throughput microscopy and in-situ sequencing. Another limitation of PE tagging is the size of the tag for insertion, which is much more restricted than intron tagging. In order to adapt this approach to scalable recruitment, a split HaloTag which was recently developed, may be used as a donor tag.

### Example 3—Identification of Degradation Competent E3 Ligases

Provided herein is an E3 ligase-wide profiling technology based on endogenous tagging of every postulated E3 ligase with bio-orthogonal tags which can be used to probe the recruitability of each ligase to a model substrate in a multiplexed fashion. This approach, called Ligase Mapping (Lig-Map) (FIG. 1), enables the generation of a global profile of degradation competent E3 ligases, across multiple cellular localizations. This platform outputs the potential effectors/target pair and information about the potential optimal linker length and effector/target configuration/orientation, which provides a starting point for subsequent med chem development of small molecules that will bring that specific effector/target pair together.

This profiling technology uses chemically induced dimerization approaches in concert with endogenous tagging to determine which of the >800 predicted E3 ligases from the human genome are able to degrade a neosubstrate, thus making them suitable for PROTAC recruitment. Two clonal cell lines have been generated, which express eGFP-FKBP fusion with and without an NLS attached to the GFP. Testing the degradation of a GFP localized to the nucleus is important, as many oncology targets are transcription factors which might not be efficiently degraded by cytoplasmically localized E3 ligases. Using these two genetic backgrounds, all expressed E3 ligases will be endogenously tagged with a HaloTag domain such that they can be recruited to the eGFP-FKBP and potentially induce its degradation upon addition of the chemical dimerizer. Endogenous tagging will be performed using intron tagging in two steps. In the first step, a large intronic sgRNA library will be made, using all introns for each gene with four sgRNAs in each. This library will be separated into three sub libraries according to the coding frame of each intron with a corresponding matching donor. Tagged libraries will be treated with a HaloTag TMR ligand, which is used to sort out cells with an expressed tagged protein. Then, the localization patterns of each tagged protein will be analyzed with high throughput automated microscopy and in-situ sequencing as described above to filter out diffuse localization patterns (suggesting an effect on the folding of the tagged protein) and outlier intronic localization patterns (cases where a minority of the tagged introns show a different localization pattern). At this point, depending on the skew and complexity of the sorted cell library, a new library may be reconstructed using 2-3 validated sgRNAs for each effector gene.

Once the pooled cell library has been prepared and characterized, cell populations will be treated with 2 doses (100 nM and 10 μM) of three different chemical dimerizers. Two doses are necessary since the compounds inducing chemical dimerization are prone to the so-called “hook effect” whereby at increased concentrations, non-productive dimers form preferentially over productive ternary complexes. Additionally, three different compounds spanning different linker lengths between the FKBP ligand and the HaloTag ligand will be sued due to the importance of linker length and composition in ternary complex formation. The combination of 2 doses and 3 linkers is expected to be sufficient to efficiently capture the majority of possible interactions that may result in eGFP-FKBP degradation and better understand the effect of dose and linker length on degradation. Following treatment, the cell populations will be incubated for 12 hours (sufficient time for protein degradation to occur in most cases) before being sorted by fluorescence-activated cell sorting (FACS). Cells with reduced GFP fluorescence will be collected and undergo next generation sequencing analysis to identify effectors capable of inducing eGFP degradation in a compound specific fashion (FIG. 11). As sgRNAs for tagging are introduced into cells using low MOI lentiviral libraries, readout and analysis of the sequencing data will follow standard CRISPR screening protocols.

To confirm hits from this screen, clonal populations of effector tagged cells will be derived using a similar methodology to as described above and the protein degradation phenotype confirmed by flow-cytometry and immuno-blotting analysis over a higher resolution dose response and time course. Additionally, organelle specificity will be confirmed by tagging the same effector in the two different eGFP-FKBP localized cell lines. To demonstrate generality of E3 ligase recruitment, the degradation induced by effector recruiting to a different substrate, BRD4, will be validated via immunoblotting. BRD4 will be employed as it has become the key confirmatory target for new E3 ligase application in targeted protein degradation and ligands are readily available for incorporation into HaloTag bifunctional dimerizers (FIG. 2D) enabling the validation without derivation of additional cell lines.

This approach is expected to provide a shortlist of E3 ligases capable of inducing ectopic ubiquitination and protein degradation in a heterobifunctional compound specific fashion. These data will greatly enhance the understanding of the effector opportunities with the E3 ligase super family as well as provide a rational decision-making tool for E3 ligase ligand development efforts by the medicinal chemistry community.

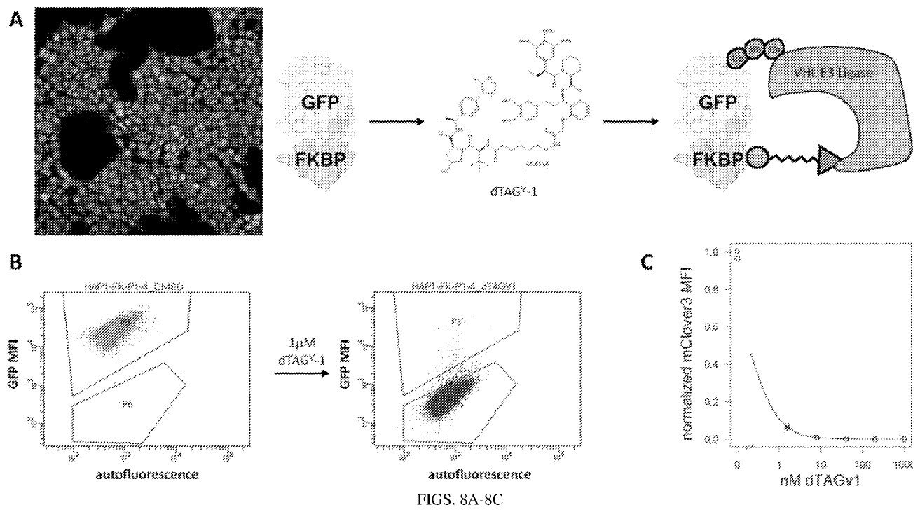

A model target protein, eGFP-FKBP fusion, has been generated and tested (FIGS. 8A-8C). FIG. 8A shows a confocal image and overview of a model target protein in HAP1 cells. A GFP (in this case mClover3) is fused with a ligand binding domain (FKBPF36V) and uniformly expressed throughout the cytoplasm. The susceptibility of the target to degradation was tested with dTAGv-1, which binds to both the FKBP domain and the VHL E3 ligase (PMID 32948771). FIG. 8B shows the flow cytometry a clonal fluorescent cell line (pictured in FIG. 8A) treated with 1 μM dTAGv-1 for 22 hours. FIG. 8C shows the dose response of dTAGv-1 after 23 hours of treatment.

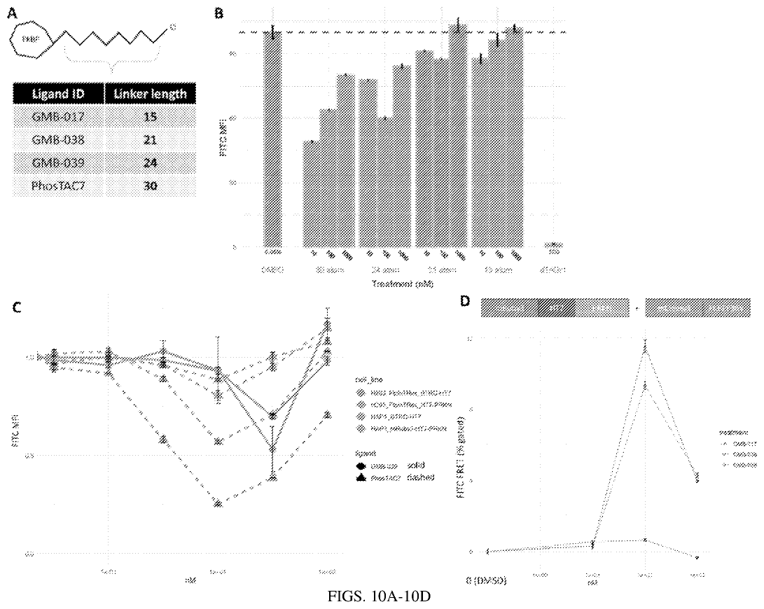

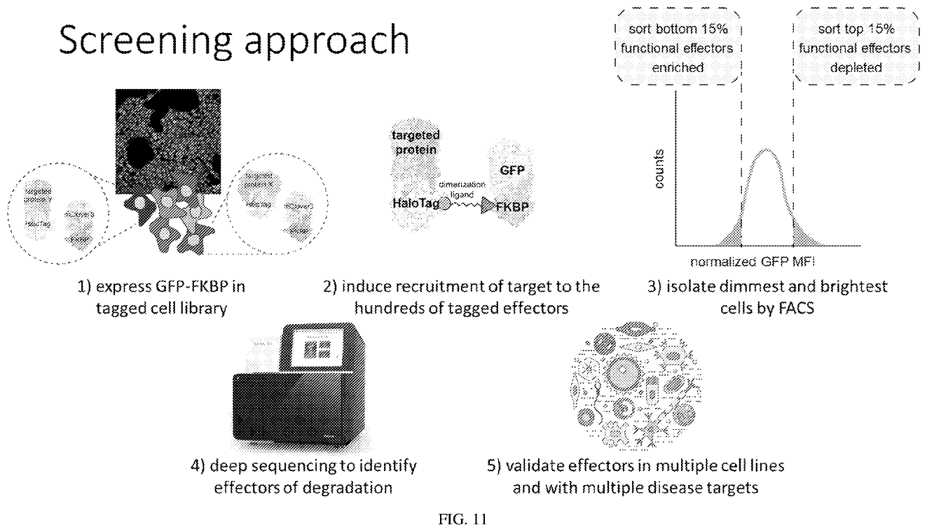

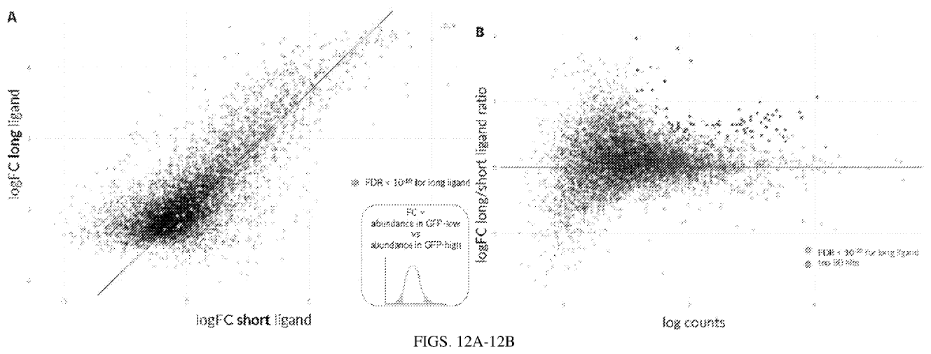

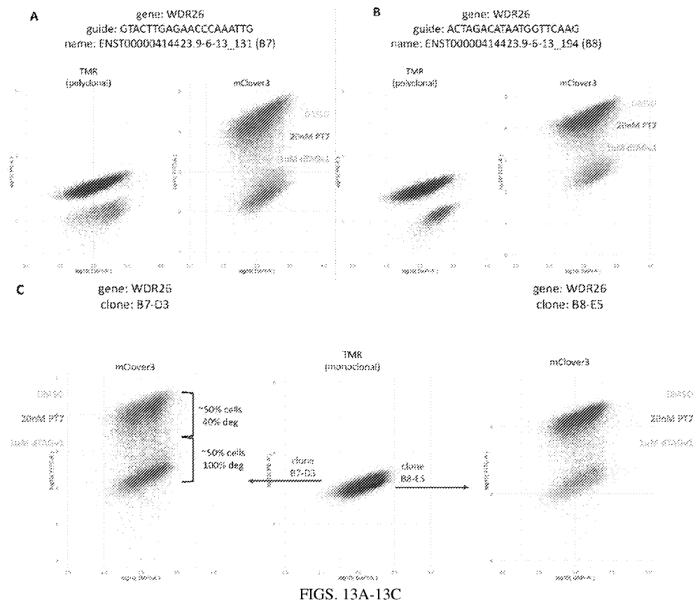

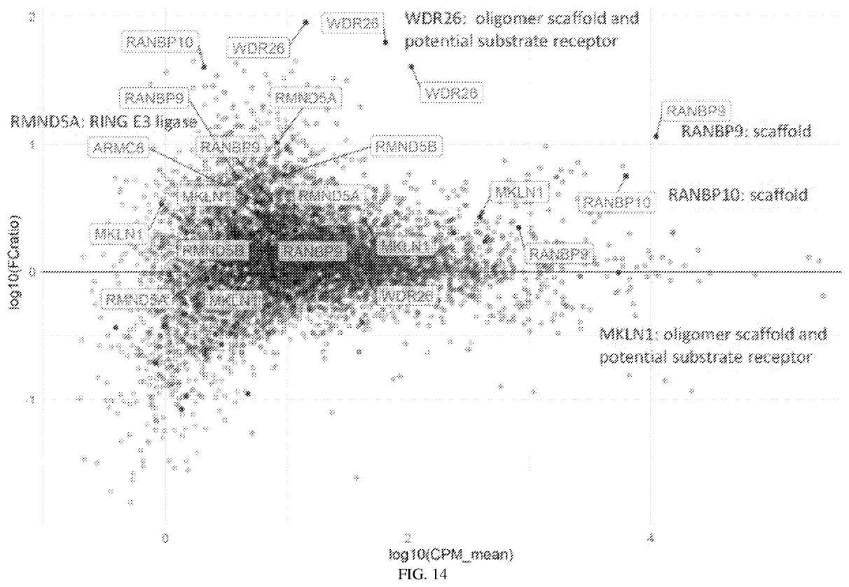

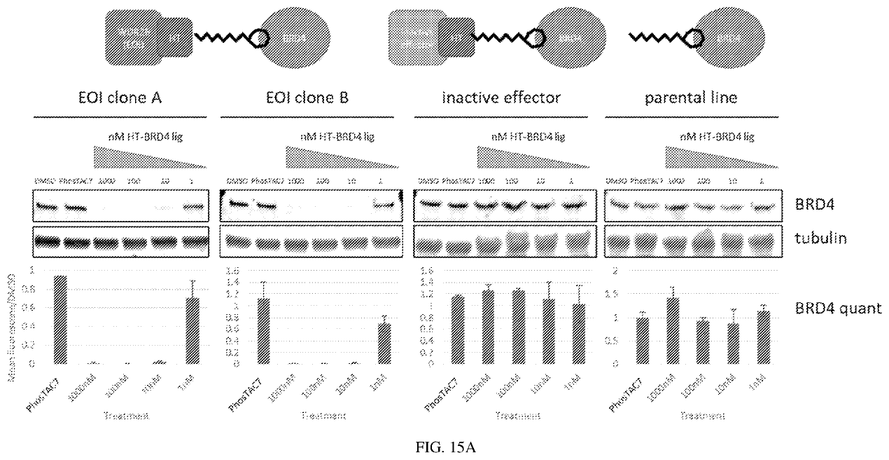

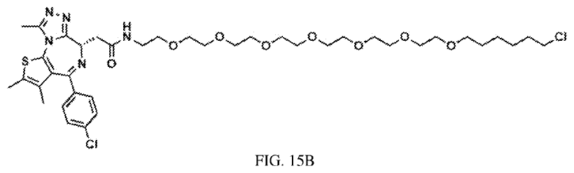

A toolbox of generic ligands has been tested (FIGS. 10A-10D). A schematic of generic ligands targeting both FKBPF36V and HaloTag of various linker lengths is provided in FIG. 10A. Ligands with various linker lengths were used to treat HEK 293 cells expressing both EGFP-FKBPF 36V and BTRCP-HT (PMID 28767222) for 23 hours. 0.5 μM dTAGv-1 was used as a positive control. Ligands with longer linkers are more potent in this model (FIG. 10B). FIG. 10C provides the 23 hour dose response of GMB-039 and PhosTAC7 (PMID 34780684) in various cell lines. Cell lines include HEK 293s with EGFP-FKBPF36V and BTRCP-HT or HT-PRKN, and HAP1s with mClover3-FKBPF36V and BTRCP-HT or mRuby3-HT7-PRKN. FIG. 10D provides a schematic of the HAP1 line expressing mClover3-FKBPF36V and mRuby3-HT7-PRKN for FRET analysis in response to various doses of the generic ligands. Longer ligands induce much stronger FRET than shorter ligands.

### Example 4—Identification of a Suitable E3 Ligase for a Target

The Lig-Map approach can be employed to rapidly identify promising E3 ligases for therapeutically relevant proteins across proteins and cell models. To exemplify this approach, two targets will be used, one which has been validated as a PROTAC target previously and one with therapeutic potential but no reported PROTAC.

Initially, the E3 ligases capable of inducing the degradation of BCR-Abl, the oncogenic driver in chronic myeloid leukemia which results from the Philadelphia chromosome, will be screened. Multiple PROTACs which induce the degradation of BCR-Abl via either CRBN, CIAP, RNF114 or VHL ligases have been reported. As such, the re-identification of these ligases will serve as a positive control within the tagged ligase library, as well as the identification of ligases which have not previously been used. Since various small molecule ligands for endogenous BCR-Abl protein are available, a single tag approach will be used, whereby the endogenous ligases are tagged as described above in K562 cells and a series of Asciminib-HaloTag heterobifunctional compounds (FIG. 2E) employed to induce degradation. In this case, the Asciminib-HaloTag ligand alone will have antiproliferative effects on K562 cells due to inhibition of oncogenic BCR-Abl signaling, however BCR-Abl PROTACs have been shown to be antiproliferative at lower concentrations due to the simultaneous abrogation of scaffolding roles. A concentration at which the heterobifunctional compounds are non-toxic (e.g., IC10 in antiproliferative assays) in untagged K562 cells will be determined. This concentration will be used for screening in the endogenously tagged cell library. Degradation of BCR-Abl is expected to lead to significant inhibition of cell proliferation such that the E3 ligases inducing degradation will drop out of the cell pool over a 4-week period of expansion in the presence of dimerizer. To assess which ligases are depleted from the cell pool, next-generation sequencing (NGS) of bulk amplicon of the sgRNA will be performed weekly.

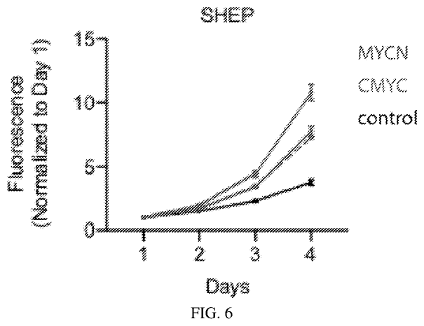

Concurrently, the Lig-Map approach was also applied to MYCN, a member of the Myc family of transcription factors that is frequently amplified or overexpressed in various cancers, especially neuroblastoma. Exogenous expression of MYCN or CMYC using a lentiviral vector in SHEP neuroblastoma cells results in fast proliferation providing a simple model for MYCN driven cancer (FIG. 6). This model will be used and MYCN fused to FKBP will be expressed followed by LigMap tagging of E3 ligases. After adding chemical dimerizers, E3 ligases that are able to degrade MYCN are expected to counter the fast proliferation and to start to drop out of the cell pool. These cells will be grown over a period of four weeks in the presence of the chemical dimerizer and bulk amplicon NGS of the sgRNA will be performed in weekly time points to identify those sgRNAs that continuously deplete out of the cell pool, suggesting degradation of the oncogenic protein.

Following the Lig-Map based identification of BCR-Abl and MYCN degrading ligases, careful control experiments will be performed to validate the candidate ligases. Initially clonal populations of cells expressing HaloTag candidate ligase fusion proteins will be established using a targeted endogenous tagging approach. These cell lines will be used to confirm that each candidate ligase can indeed induce degradation of the target protein in a compound specific fashion by immunoblotting analysis. At this stage, a des-chloro control negative control compound, incapable of HaloTag engagement will be used to confirm that degradation is dependent on dimerization. Furthermore, that degradation of the target protein occurs via the anticipated ubiquitin proteasome system mechanisms will be validated. For example, co-treatment of cells with a proteasome inhibitor would be anticipated to abrogate protein degradation, as would inhibition of neddylation for the Cullin ring ligases. Similarly, qPCR analysis will be performed to confirm that the loss of protein is occurring post-translationally. Together these control experiments will validate candidate E3 ligases as bona fide PROTAC compatible proteins.

This set of experiments will not only provide a useful strategy/starting point for the development of therapeutic approaches to BCR-Abl and MYCN degradation (potentially using previously published recruiting elements) but also provide a key proof of principle for the Lig-Map platform and its ability to rapidly generate favourable E3 ligase/neo-substrate pairs. The ability to rapidly identify the most amenable E3 ligase for any given protein will greatly enhance the ability to develop targeted protein degradation strategies and therefore potentially provide therapeutic strategies for a multitude of diseases.

### Example 5—F—Map Platform Validation

One conceptual advance from the F-Map approach is the discovery of therapeutically relevant effectors in an unbiased fashion. Screening all possible effector proteins will provide for the discovery of induced protein-protein interactions with a desirable cellular phenotype, without a prior understanding of the biology required with other therapeutic strategies (e.g., inhibitor development). To demonstrate this, two model systems will be used for which induced proximity of an effector protein may provide a therapeutic benefit: First, the F-Map approach will be used to identify effectors that increase membrane expression of CFTR in cystic fibrosis and secondly, the F-Map approach will be used to identify effectors which can prevent and even reverse mHTT aggregation in iPSC derived neurons.

The cystic fibrosis transmembrane conductance regulator (CFTR) protein is a chloride channel, mutation of which causes cystic fibrosis. The most common mutation is AF508, resulting in a protein with an unstable fold which gets ubiquitinated and degraded before ever reaching the plasma membrane, thus impairing chloride homeostasis and resulting in the disease phenotype. Recent studies have demonstrated that recruitment of deubiquitinating enzymes (DUBs) via chemical dimerization or nanobody targeting can stabilize CFTR and thus provide a therapeutic strategy beyond currently employed pharmacological chaperones. However, these studies only recruited a single DUB, dictated by the availability of a chemical handle for recruitment. The F-Map platform will enable the discovery of the optimal effector protein to stabilize CFTR from a panel of diverse effectors.

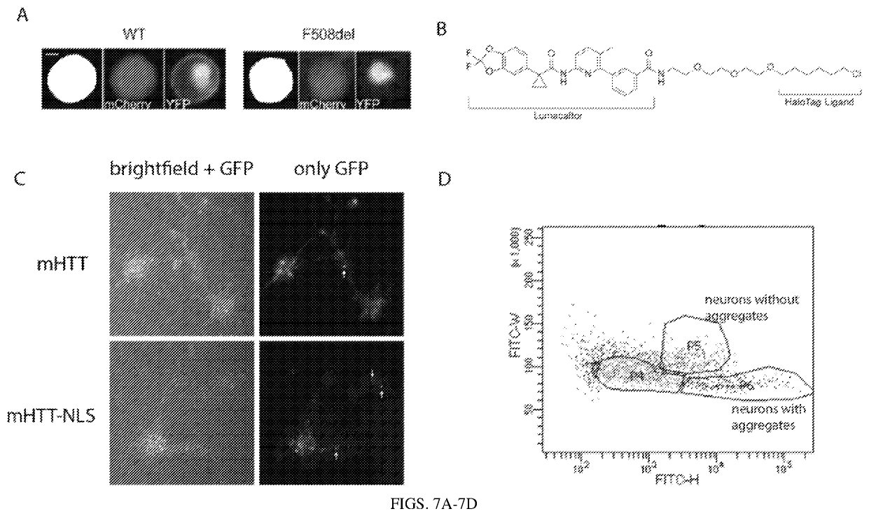

To measure AF508 CFTR membrane proximity rescue, a fluorescence imaging approach has been coupled to in-situ sequencing to identify the tagged proteins. HEK293 cells have been transduced with lentivirus encoding pIRES2-mCherry-YFP-F508del-CFTR resulting in cells expressing cytosolic mCherry and ER localized YFP-F508del-CFTR (FIG. 7A). Treatment with pharmacological chaperone Lumacaftor restores a membrane proximal YFP signal (observed with wt-YFP-CFTR) (FIG. 7A). These cells were then transduced with the F-Map tagging library and treated with the chemical inducers of proximity derived from Lumacaftor and HaloTag recruiting chloroalkane ligands (FIG. 7B). High-throughput automated microscopy, enabled by the cytosolic mCherry defining cell outlines, was used to identify cells with membrane proximal CFTR and in-situ sequencing will be used to identify the tagged protein responsible for CFTR rescue. The identification of DUBs is expected, but also potentially chaperones or acetyltransferase enzymes which may enforce stable protein folding or compete for ubiquitination at lysine residues specifically. The identified effectors will be validated using similar approaches described above, followed by confirmation that CFTR function is restored by electrophysiology through collaborations.

A hallmark of Huntington Disease (HD) and most other neurodegenerative diseases is the presence of toxic protein aggregates in neurons resulting in neuronal death and disease. HD is caused by a polyglutamine expansion in the protein Huntingtin (HTT) causing the formation of intranuclear inclusion bodies which are suggested to drive disease in vulnerable neurons. F-map will be used to identify effector proteins that will be able to prevent and even reverse the formation of these aggregates. A similar approach can be applied to many additional neurodegenerative diseases in which the formation of protein aggregates is a major hallmark. To test the ability of the F-map platform to identify effector proteins that can modify the formation and even disassemble protein aggregates in a relevant cell model, an iPSC-derived Ngn2 rapid differentiation model, where a mHTT N terminal fragment is inducibly expressed and forms nuclear and cytoplasmic aggregates in mature neurons, will be used (FIG. 7C). Effector tagging will be done in the iPSC stage. PE tagging will also be tested in iPSC by delivering the PE enzymes using a piggypac transposases, and pegRNA libraries with low MOI lentiviral vectors. Two time points will be used to add the chemical dimerizer, one together with expression of mHTT, to identify effector proteins that would prevent formation of aggregates, and the second after the number of aggregates has maxed out (two weeks after induction) to look for effectors that are able to disassemble these inclusions. Two approaches will be used for comparing effector distribution between cells with and without aggregates in order to identify effector proteins that either positively or negatively affect aggregation. The first approach will be imaging based profiling and in-situ sequencing, since this does not require segmentation of the full neuron which might be challenging, only the soma and nucleus. As an alternative to the image-based approach, FACS-based sorting may be used. PulSA is a FACS approach that can be used to identify cells with aggregates (FIG. 7D). Chaperons and other components of protein quality control and protein degradation machinery are expected to be identified. Two aspects of this experiment make it extremely important for improving the understanding of basic cell biology. First, as protein aggregates can be formed in the nucleus, the F-Map system will enable the study of nuclear protein degradation which is much less studied and understood in comparison with degradation of proteins in the cytoplasm. Second, while proteins with disaggregase activity are known in yeast (e.g., HSP104), it is unclear if there are human proteins that harbor the same capabilities. By inducing proximity after aggregate formation, proteins with such capabilities will be able to be identified.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Wang et al., “Targeted protein acetylation in cells using heterobifunctional molecules,” J. Am. Chem. Soc., 143:16700-16708, 2021.
Siriwardena et al., “Phosphorylation-inducing chimeric small molecules,” J. Am. Chem. Soc., 142:14052-14057, 2020.
Chen et al., “Modulation of phosphoprotein activity by phosphorylation targeting chimeras (ProTACs)”, ACS Chem. Biol., 16:2808-2815, 2021.

What is claimed is:

1. A method of identifying a target/effector protein pair, wherein the effector protein has a desired effect on the target protein following chemical induction of dimerization, the method comprising:
(i) obtaining a population of cells in which the target protein comprises or is tagged with a ligand binding domain;
(ii) endogenously tagging potential effector proteins with orthogonal ligand binding domains, such that each cell in the population of cells comprises a single effector protein tagged with an orthogonal ligand binding domain and an integrated molecular barcode;
(iii) contacting the population of cells with a heterobifunctional molecule that binds to both the ligand binding domain and the orthogonal ligand binding domain, thereby inducing protein-protein interactions between the target protein and the potential effector proteins; and
(iv) identifying the potential effector proteins that induce the desired effect on the target protein by selecting cells with a phenotype of interest.

2. The method of claim 1, wherein the ligand binding domain with which the target protein is endogenously tagged is FKBP^{F36V}.

3. The method of claim 1 or 2, wherein the orthogonal ligand binding domain with which the potential effector proteins are tagged are HaloTags, split HaloTags, or SNAP-Tags.

4. The method of any one of claims 1-3, wherein the phenotype of interest is the target protein expression level, altered growth rate, cell abundance, target protein disaggregation, target protein stabilization, or target protein localization.

5. The method of any one of claims 1-4, wherein step (iv) comprises identifying the molecular barcode in the cells displaying the phenotype of interest, thereby identifying the effector proteins present in the selected cells.

6. The method of any one of claims 1-5, wherein after step (ii), tagging of potential effector proteins is confirmed using high-throughput imaging and/or in-situ barcode sequencing.

7. The method of any one of claims 1-6, wherein step (ii) comprises integrating an exogenous DNA sequence into an intronic genomic sequence in various cells of the population, wherein the intronic genomic sequence is unique for each of the cells, wherein said integrating is achieved by delivering to the cell population (a) a library of sgRNAs designed to target introns of the effector genes; and (b) a composition comprising (i) an endonuclease-encoding nucleic acid sequence, (ii) a donor plasmid comprising nucleic acid comprising, from 5′ to 3′, a first sgRNA binding site, a splice acceptor site, a sequence encoding the orthogonal ligand binding domain, a splice donor site, and a second sgRNA binding site, and (iii) a donor plasmid-specific gRNA-encoding sequence.

8. The method of claim 7, wherein the endonuclease is a Cas endonuclease.

9. The method of claim 8, wherein the Cas endonuclease is a Cas9 endonuclease.

10. The method of claim 7, further comprising a site-specific guide RNA (gRNA)-encoding nucleic acid sequence.

11. The method of claim 7 or 10, wherein the site-specific and/or donor plasmid-specific guide RNA is a single gRNA.

12. The method of claim 11, wherein the site-specific and/or donor plasmid-specific guide RNA is a CRISPR-RNA (crRNA).

13. The method of claim 12, wherein the site-specific and/or donor plasmid-specific guide RNA comprises a fusion of a crRNA and a trans-activating CRISPR RNA (tracrRNA).

14. The method of claim 7 or 10, wherein the guide RNA comprises a crRNA and a tracrRNA.

15. The method of claim 7, wherein the endonuclease and the donor plasmid-specific gRNA are encoded on a single nucleic acid molecule.

16. The method of claim 7, wherein the endonuclease, the donor plasmid-specific gRNA, and the donor plasmid are encoded on a single nucleic acid molecule.

17. The method of claim 7 or 10, wherein each of the endonuclease-encoding nucleic acid sequence, the donor plasmid, the site-specific guide RNA (gRNA)-encoding nucleic acid sequence, and the donor plasmid-specific gRNA-encoding sequence are present on separate nucleic acid molecules.

18. The method of any one of claims 7-17, wherein the first and second sgRNA binding sites of the donor plasmid comprise the same nucleotide sequence.

19. The method of any one of claims 7-18, wherein the donor plasmid further comprises an antibiotic resistance gene positioned between the splice donor site and the second sgRNA binding site or between the first sgRNA binding site and the splice acceptor site.

20. The method of claim 19, wherein the antibiotic resistance gene is blasticidin.

21. The method of any one of claims 7-20, wherein the method comprises using a library of sgRNAs that target all introns of the effector genes.

22. The method of any one of claims 7-21, wherein the method comprises using a library of sgRNAs that correspond to all three coding frames.

23. The method of any one of claims 7-22, wherein the portion of the donor plasmid comprising the splice acceptor site, the sequence encoding the orthogonal ligand binding domain, and the splice donor site is integrated into the intronic genomic sequence of a target gene.

24. The method of any one of claims 7-23, wherein the orthogonal ligand binding domain is expressed when the target gene is expressed.

25. The method of claim 19 or 20, further comprising detecting the expression of the antibiotic resistance gene.

26. The method of any one of claims 18-25, further comprising detecting the expression of the orthogonal ligand binding domain.

27. The method of any one of claims 1-6, wherein step (ii) comprises integrating an exogenous DNA sequence into a genomic sequence of a gene in a cell, such that the exogenous DNA sequence will form either an N-terminal or C-terminal tag on a protein encoded by the gene, by delivering to the cell population a composition comprising (a) prime editing enzymes and (b) a library of pegRNAs comprising the orthogonal ligand binding domain.

28. The method of any one of claims 1-27, wherein the target gene is an endogenous gene.

29. The method of any one of claims 1-28, wherein the target gene is BCR-Abl, CFTR, MYCN, or mHTT.

30. The method of any one of claims 1-27, wherein the target gene is an exogenous fluorescent protein.

31. The method of claim 30, wherein the exogenous fluorescent protein is GFP.

32. The method of any one of claims 1-31, wherein the effector proteins are E3 ligases.

33. The method of any one of claims 1-32, wherein the effector proteins are kinases, phosphatases, E3 ligases, chaperones, deubiquitinating enzymes, methyl transferases, demethylases, acetyltransferase, deacetylating enzymes, glycosyltransferases, and dissagregases.
